SEQUENCE LISTING (14 sequences)

SEQ ID NO: 1 - DNA, 18 bp, synthetic construct
  misc_feature 1..18: note = sequence of forward primer for beta-actin
  source 1..18: mol_type = other DNA
taggcaccagggtgtgat

SEQ ID NO: 2 - DNA, 16 bp, synthetic construct
  misc_feature 1..16: note = sequence of reverse primer for beta-actin
  source 1..16: mol_type = other DNA
ctcctcaggggccaca

SEQ ID NO: 3 - DNA, 23 bp, synthetic construct
  misc_feature 1..23: note = sequence of forward primer for Fn-1
  source 1..23: mol_type = other DNA
gacgaagagcccttacagttcca

SEQ ID NO: 4 - DNA, 20 bp, synthetic construct
  misc_feature 1..20: note = sequence of reverse primer for Fn-1
  source 1..20: mol_type = other DNA
tctgcagtgcctccactatg

SEQ ID NO: 5 - DNA, 19 bp, synthetic construct
  misc_feature 1..19: note = sequence of forward primer for Col-I
  source 1..19: mol_type = other DNA
cctggtccctctggaaatg

SEQ ID NO: 6 - DNA, 20 bp, synthetic construct
  misc_feature 1..20: note = sequence of reverse primer for Col-I
  source 1..20: mol_type = other DNA
ggaagcctctttctcctctc

SEQ ID NO: 7 - DNA, 22 bp, synthetic construct
  misc_feature 1..22: note = sequence of forward primer for Il-1beta
  source 1..22: mol_type = other DNA
caagcttccttgtgcaagtgtc

SEQ ID NO: 8 - DNA, 21 bp, synthetic construct
  misc_feature 1..21: note = sequence of reverse primer for Il-1beta
  source 1..21: mol_type = other DNA
ttcatcttttggggtccgtca

SEQ ID NO: 9 - DNA, 21 bp, synthetic construct
  misc_feature 1..21: note = sequence of forward primer for Il-6
  source 1..21: mol_type = other DNA
ttcctctctgcaagagacttc

SEQ ID NO: 10 - DNA, 20 bp, synthetic construct
  misc_feature 1..20: note = sequence of reverse primer for Il-6
  source 1..20: mol_type = other DNA
gttgggagtggtatcctctg

SEQ ID NO: 11 - DNA, 20 bp, synthetic construct
  misc_feature 1..20: note = sequence of forward primer for Tnf-alpha
  source 1..20: mol_type = other DNA
agacaccatgagcacagaaa

SEQ ID NO: 12 - DNA, 20 bp, synthetic construct
  misc_feature 1..20: note = sequence of reverse primer for Tnf-alpha
  source 1..20: mol_type = other DNA
cacttggtggtttgtgagtg

SEQ ID NO: 13 - DNA, 20 bp, synthetic construct
  misc_feature 1..20: note = sequence of forward primer for Hck
  source 1..20: mol_type = other DNA
tcctccgagatggaagcaag

SEQ ID NO: 14 - DNA, 21 bp, synthetic construct
  misc_feature 1..21: note = sequence of reverse primer for Hck
  source 1..21: mol_type = other DNA
acagtgcgaccacaatggtat

(19) United States
(12) Patent Application Publication
(10) Pub. No.: US 2025/0228856 A1
(43) Pub. Date: Jul. 17, 2025

(54) USE OF HEMATOPOIETIC CELL KINASE (HCK) INHIBITOR IN PREPARATION OF DRUG FOR TREATING PULMONARY FIBROSIS

(71) Applicant: Institute of Basic Medical Sciences, CAMS, Beijing (CN)

(72) Inventors: Jing Wang, Beijing (CN); Meiyue Song, Beijing (CN); Jiaxin Wang, Beijing (CN); Wei Zeng, Beijing (CN)

(73) Assignee: Institute of Basic Medical Sciences, CAMS, Beijing (CN)

(21) Appl. No.: 18/740,951

(22) Filed: Jun. 12, 2024

(30) Foreign Application Priority Data
Jan. 12, 2024 (CN) 202410053142.8

(51) Int. Cl. A61K 31/519; A61K 38/46; A61K 38/48; A61P 11/00

(52) U.S. Cl. CPC A61K 31/519; A61K 38/465; A61K 38/48; A61P 11/00

(57) ABSTRACT

The present disclosure provides use of a hematopoietic cell kinase (HCK) inhibitor in preparation of a drug for treating pulmonary fibrosis, and relates to the technical field of biomedicine. In the present disclosure, idiopathic pulmonary fibrosis (IPF) model mice are used as a subject to allow research. Administration of an HCK inhibitor RK-20449 to IPF mice can effectively alleviate the progression of pulmonary fibrosis. A weight and a survival rate of the IPF mice have improved to a certain extent, and their lung function has improved significantly. The transcription levels of inflammatory factors Il-1β, Il-6, and Tnf-α in a lung tissue of the IPF mice have decreased, while the concentrations of inflammatory factors IL-1β and IL-6 in the alveolar lavage fluid have also decreased. The transcription levels of fibrosis factors Col-I and Fn-1 are decreased, the collagen-specific amino acid hydroxyproline is decreased, and the degree of lesions is alleviated.

### CROSS REFERENCE TO RELATED APPLICATION

This patent application claims the benefit and priority of Chinese Patent Application No. 2024100531428, filed with the China National Intellectual Property Administration on Jan. 12, 2024, the disclosure of which is incorporated by reference herein in its entirety as part of the present application.

### REFERENCE TO SEQUENCE LISTING

A computer readable XML file entitled “GWP20231209466_sequence listing.xml”, which was created on Mar. 5, 2024 with a file size of about 18,924 bytes, contains the sequence listing for this application, has been filed with this application, and is hereby incorporated by reference in its entirety.

### TECHNICAL FIELD

The present disclosure belongs to the technical field of biomedicine, and specifically relates to use of a hematopoietic cell kinase (HCK) inhibitor in preparation of a drug for treating pulmonary fibrosis.

### BACKGROUND

Interstitial lung disease (ILD) is a general term for a group of diffuse pulmonary diseases involving the pulmonary interstitium, alveoli, and/or bronchioles. The ILD has many classifications and complex causes. Idiopathic pulmonary fibrosis (IPF) is the most representative ILD. The IPF is more likely to occur in middle-aged and elderly people with an unclear cause, and is characterized by progressive and irreversible pulmonary parenchymal fibrosis, leading to a progressive decline in lung function. The incidence of IPF has shown a significant upward trend in recent years. At the same time, a median survival period of IPF patients after diagnosis is only 2 to 5 years, and a 5-year survival rate is less than 30%. Therefore, the IPF becomes a severe and fatal pulmonary fibrosis disease, known as “a cancer that is not cancer.” Since the pathogenesis of IPF is extremely complex and involves a wide range of factors, no specific treatment drug has been found so far, and there is a lack of clear treatment basis. Accordingly, in-depth exploration of the pathogenesis of IPF and the search for more effective new drug targets can provide the best scientific basis for targeted treatment of IPF.

A series of recent studies have reported that abnormalities in SRC family kinases can lead to the progression of various diseases, including inflammation, autoimmune diseases, and cancer. The SRC family is a class of non-receptor tyrosine kinases, containing about 9 members, such as Lck, Blk, and Hck. The SRC family can be divided into two major categories based on different expression patterns, where three members Src, Yes, and Fyn, are widely expressed in vivo, while the other six members are expressed in specific tissue cells. Hematopoietic cell kinase (HCK) is mainly expressed in two subtypes (human p61 and p59, mouse p59 and p56) in myeloid cells or a small number of lymphoid cells, and participates in immune and other related functional activities. After being activated, the HCK can catalyze the phosphorylation of various substrates such as Bcr/Abl, Tel/Abl, PAG, and STAT5, and then participate in cell proliferation, migration, apoptosis and other activities. At present, the mechanism of action of HCK in pulmonary fibrosis has not been fully elucidated, and further research is urgently needed to explore its role in pulmonary fibrosis and possible treatment strategies.

### SUMMARY

In view of this, an objective of the present disclosure is to provide use of an HCK inhibitor in preparation of a drug for treating pulmonary fibrosis. An HCK inhibitor RK-20449 can effectively improve pulmonary dysfunction, pulmonary inflammation, and pulmonary fibrosis.

To achieve the above objective, the present disclosure provides the following technical solutions:

The present disclosure provides use of an HCK inhibitor in preparation of a drug for treating pulmonary fibrosis.

Preferably, the HCK inhibitor is one or two selected from the group consisting of a modulator capable of reducing an HCK expression level and a modulator capable of reducing an HCK product.

More preferably, the modulator capable of reducing the HCK expression level includes RK-20449.

More preferably, an RK-20449 solution has a concentration of 5 mg/mL to 15 mg/mL.

More preferably, the RK-20449 solution is prepared with phosphate-buffered saline (PBS) as a solvent.

More preferably, the HCK inhibitor includes a protease and a nuclease that degrade the HCK product.

More preferably, the drug ameliorates pulmonary dysfunction.

More preferably, the drug ameliorates pulmonary inflammation and pulmonary fibrosis.

The present disclosure further provides a drug for treating pulmonary fibrosis, including an active ingredient and a pharmaceutically acceptable carrier, where the active ingredient includes the modulator RK-20449 that reduces the HCK expression level.

Preferably, a dosage form of the drug is selected from the group consisting of a capsule, a powder, a tablet, and a solution.

Compared with the prior art, the present disclosure has the following beneficial effects:

The present disclosure provides use of an HCK inhibitor in preparation of a drug for treating pulmonary fibrosis. In the present disclosure, IPF model mice are used as a subject to allow research, and the HCK inhibitor RK-20449 is administered to the IPF mice. This significantly inhibits the transcription level of Hck in the lung tissue of IPF mice and can effectively alleviate the progression of IPF. Specifically, after giving the HCK inhibitor RK-20449, the weight of IPF mice is increased and the survival rate is also improved to a certain extent. Lung function has improved significantly, including lung volume indicators such as inspiratory capacity (IC), and pulmonary ventilation function tests such as inspiratory resistance (RI), dynamic compliance (Cdyn) and quasi-static compliance (Cchord). The transcription and translation levels of inflammatory factors IL-1β and IL-6 are decreased in IPF mice, while the transcription level of Tnf-α is also decreased. The transcription levels of fibrosis factors Fn-1 and Col-I are decreased, the collagen-specific amino acid hydroxyproline (HYP) is decreased, and the degree of lesions is alleviated. Therefore, the HCK inhibitor can be used as a new treatment strategy for pulmonary fibrosis.

### BRIEF DESCRIPTION OF THE DRAWINGS

FIG. 1 shows an influence of the HCK inhibitor RK-20449 on Hck transcription level in mouse lung tissue, ***P<0.001;

FIGS. 2A-2F show influence of the HCK inhibitor RK-20449 on the body weight, survival rate, and lung function of IPF mice, where FIG. 2A is the weight change rate of the mice; FIG. 2B is the survival rate of mice; FIG. 2C is the RI; FIG. 2D is the Cdyn; FIG. 2E is the Cchord; FIG. 2F is the IC; *P<0.5, **P<0.01, ***P<0.001;

FIGS. 3A-3E show influence of the HCK inhibitor RK-20449 on the levels of inflammatory factors in IPF mice, where FIG. 3A: the transcription level of Il-1β in mouse lung tissue; FIG. 3B: transcription level of Il-6 in mouse lung tissue; FIG. 3C: Tnf-α transcript level in mouse lung tissue; FIG. 3D: IL-1β concentration in mouse bronchoalveolar lavage fluid, BALF represents bronchoalveolar lavage fluid; FIG. 3E: IL-6 concentration in mouse bronchoalveolar lavage fluid; *P<0.5, **P<0.01, ***P<0.001; and

FIGS. 4A-4C show influence of the HCK inhibitor RK-20449 on pulmonary fibrosis in IPF mice, where FIG. 4A is the transcription level of Col-I in mouse lung tissue; FIG. 4B is the transcription level of Fn-1 in mouse lung tissue; FIG. 4C is the content of hydroxyproline in mouse lung tissue; *P<0.5, **P<0.01, ***P<0.001.

### DETAILED DESCRIPTION OF THE EMBODIMENTS

The present disclosure provides use of an HCK inhibitor in preparation of a drug for treating pulmonary fibrosis. In the present disclosure, the pulmonary fibrosis mainly refers to fibrotic interstitial lung disease (f-ILD), a heterogeneous disease characterized by obvious fibrosis and inflammation in the lung interstitium. A type of the pulmonary fibrosis is not particularly limited. All types of f-ILD that can be diagnosed according to the “Expert Consensus on Diagnosis and Treatment of Interstitial Lung Disease” are within the scope of the present disclosure. Such types preferably include IPF, fibrotic non-specific interstitial pneumonia, chronic hypersensitivity pneumonitis, connective tissue disease-related interstitial lung disease, and pneumoconiosis.

In the present disclosure, the HCK inhibitor is one or two selected from the group consisting of a modulator capable of reducing an HCK expression level and a modulator capable of reducing an HCK product.

In the present disclosure, the modulator capable of reducing the HCK expression level includes RK-20449. The RK-20449 is a broad-spectrum pyrrole-pyrimidine inhibitor with high selectivity for Src kinases. There is no special limitation on a source of the HCK inhibitor RK-20449, which can be purchased through conventional commercial channels. The HCK inhibitor RK-20449 is purchased from MedChemExpress (MCE).

In the present disclosure, the RK-20449 solution has a concentration of preferably (5-15) mg/mL, more preferably (7-10) mg/mL, and even more preferably 7.5 mg/mL.

In the present disclosure, the RK-20449 solution is prepared with PBS as a solvent, specifically 7.5 mg of RK-20449 is dissolved in 1 mL of PBS solution.

In the present disclosure, the HCK inhibitor includes a protease and a nuclease that degrade the HCK product.

In the present disclosure, the drug ameliorates pulmonary dysfunction.

In the present disclosure, the drug ameliorates pulmonary inflammation and pulmonary fibrosis.

The present disclosure further provides a drug for treating pulmonary fibrosis, including an active ingredient and a pharmaceutically acceptable carrier, where the active ingredient includes the modulator RK-20449 that reduces the HCK expression level. The carrier includes a buffer, a vehicle, a stabilizer, or a preservative, for example, starch, lactose, magnesium stearate, sodium sulfite, and ascorbic acid. Routes of administration of the drug provided by the present disclosure may include oral, intravenous, parenteral, intramuscular, subcutaneous, intraperitoneal, intranasal, rectal, or topical administration. In the present disclosure, a dosage of the medicament provided by the present disclosure may be determined by disease type, disease severity, route of administration, age, gender and health conditions of patients.

In the present disclosure, a dosage form of the drug is selected from the group consisting of a capsule, a powder, a tablet, and a solution.

The technical solution provided by the present disclosure will be described in detail below with reference to the examples, but they should not be construed as limiting the claimed scope of the present disclosure.

### Example 1

The HCK inhibitor can effectively improve the body weight, survival rate, lung function, inflammation, and fibrosis of mice with pulmonary fibrosis.

### (1) Construction of an IPF Mouse Model and Administration of the HCK Inhibitor RK-20449

Male C57BL/6J mice (aged 8 weeks and weighing 25-30 g) were selected and housed in an SPF grade laboratory animal room, and the model of IPF was constructed by one-time intratracheal instillation of bleomycin (BLM); the mice were divided into four groups (n=9):
1) PBS group+vehicle group: PBS (the volume consistent with the subsequent BLM infusion volume) was given by tracheal instillation; RK-20449 solvent PBS was injected intraperitoneally;
2) PBS+RK-20449 group: intratracheal instillation of PBS (volume consistent with subsequent BLM infusion volume); intraperitoneal injection of RK-20449 (30 mg/kg, once a day, 6 d a week, for 3 weeks);
3) BLM+vehicle group: BLM solution (2 U/kg) was given tracheal infusion; RK-20449 solvent PBS was injected intraperitoneally; and
4) BLM+RK-20449 group: BLM solution (2 U/kg) was given tracheal infusion, and RK-20449 (30 mg/kg, once a day, 6 d a week, for 3 weeks) was injected intraperitoneally.

All mice were sacrificed after 21 d of administration, and the corresponding samples were collected for detection.

### (2) Mouse Weight and Survival Rate

The mice in each group were weighed, and the death of the mice was observed and counted.

### (3) Pulmonary Function Test

Anesthetized mice were fixed on an experimental table, and mouse lung function was detected by a pulmonary function testing system (DSI Buxco, USA). Before experiment, the mouse was anesthetized by intraperitoneal injection of 0.4 mL/100 g 2% pentobarbital, tracheotomy was performed, a trachea cannula was inserted, and a ventilator was connected. Next, FRC, PV, FV, and RC were automatically tested by a PET system. Indicators closely related to lung function changes in pulmonary fibrosis for statistical analysis, including lung volume indicators such as IC, and pulmonary ventilation function tests such as RI, Cdyn, and Cchord.

### (4) ELISA

Concentrations of inflammatory factors IL-1β and IL-6 in mouse BALF were detected by using ELISA kits.

(5) qPCR

Lung tissue RNA of all mice was extracted; cDNA was obtained by using a reverse transcription kit (KR103, Tiangen Biotechnology, Beijing, China); qPCR was conducted by using a SYBR Green I Q-PCR Kit (TransGen Biotech, Beijing China); data collection and analysis were conducted by Bio-Rad IQ5 system.

TABLE 1
Primer sequences (5′ to 3′)

| | | | |
|---|---|---|---|
| β-actin | F | TAGGCACCAGGGTGTGAT | SEQ ID NO: 1 |
| | R | CTCCTCAGGGGCCACA | SEQ ID NO: 2 |
| Fn-1 | F | GACGAAGAGCCCTTACAGTTCCA | SEQ ID NO: 3 |
| | R | TCTGCAGTGCCTCCACTATG | SEQ ID NO: 4 |
| Col-I | F | CCTGGTCCCTCTGGAAATG | SEQ ID NO: 5 |
| | R | GGAAGCCTCTTTCTCCTCTC | SEQ ID NO: 6 |
| Il-1β | F | CAAGCTTCCTTGTGCAAGTGTC | SEQ ID NO: 7 |
| | R | TTCATCTTTTGGGGTCCGTCA | SEQ ID NO: 8 |
| Il-6 | F | TTCCTCTCTGCAAGAGACTTC | SEQ ID NO: 9 |
| | R | GTTGGGAGTGGTATCCTCTG | SEQ ID NO: 10 |
| Tnf-α | F | AGACACCATGAGCACAGAAA | SEQ ID NO: 11 |
| | R | CACTTGGTGGTTTGTGAGTG | SEQ ID NO: 12 |
| Hck | F | TCCTCCGAGATGGAAGCAAG | SEQ ID NO: 13 |
| | R | ACAGTGCGACCACAATGGTAT | SEQ ID NO: 14 |

### (6) Detection of HYP Content

30 mg of mouse lung tissue was detected using an HYP kit (NBP2-59747, Novus Biologicals, Littleton, CO, USA).

### (7) Analysis of Results

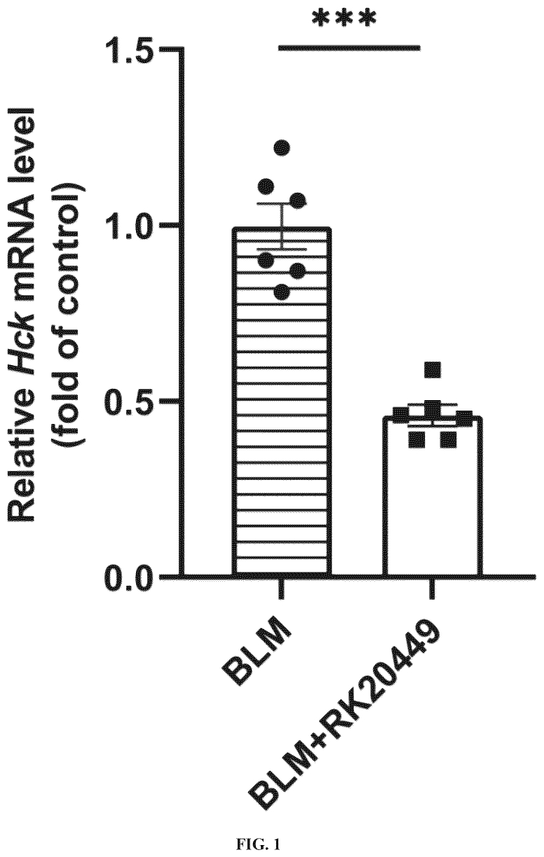

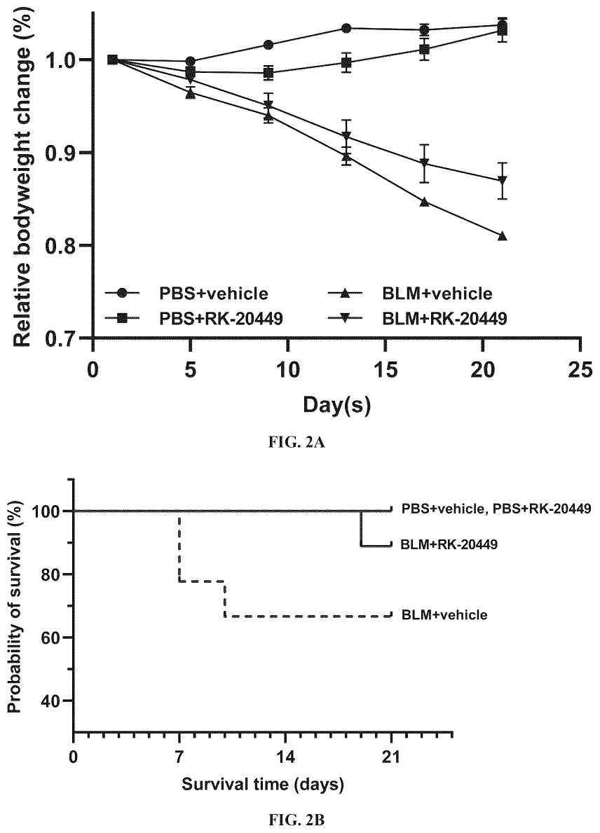

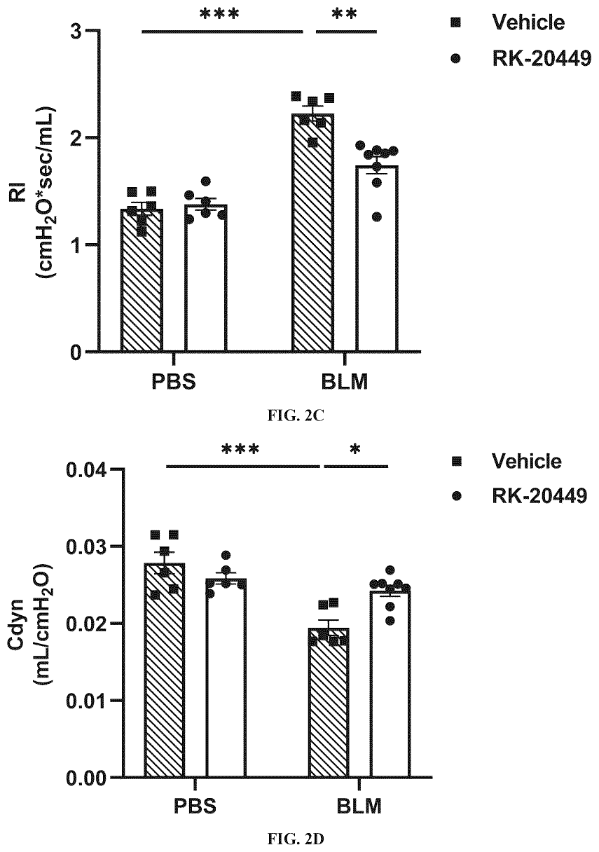

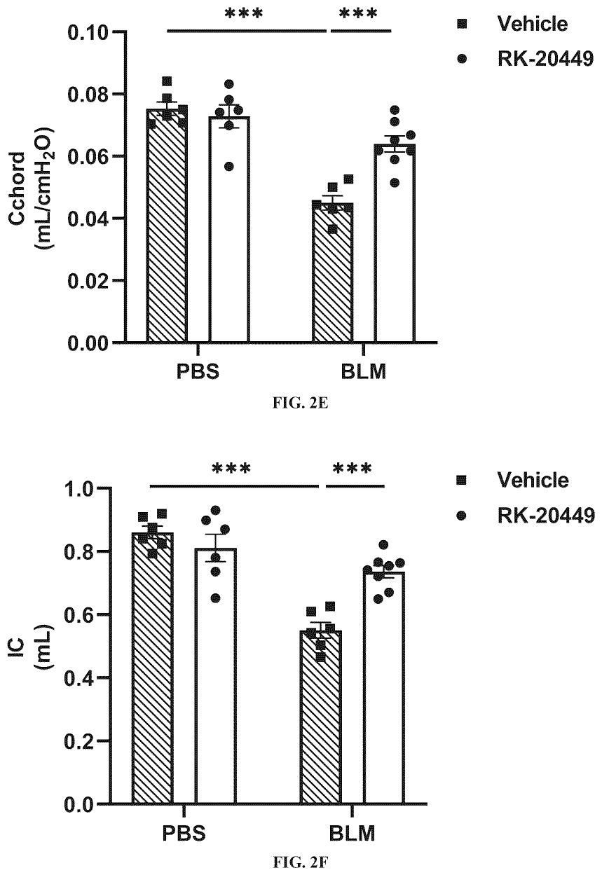

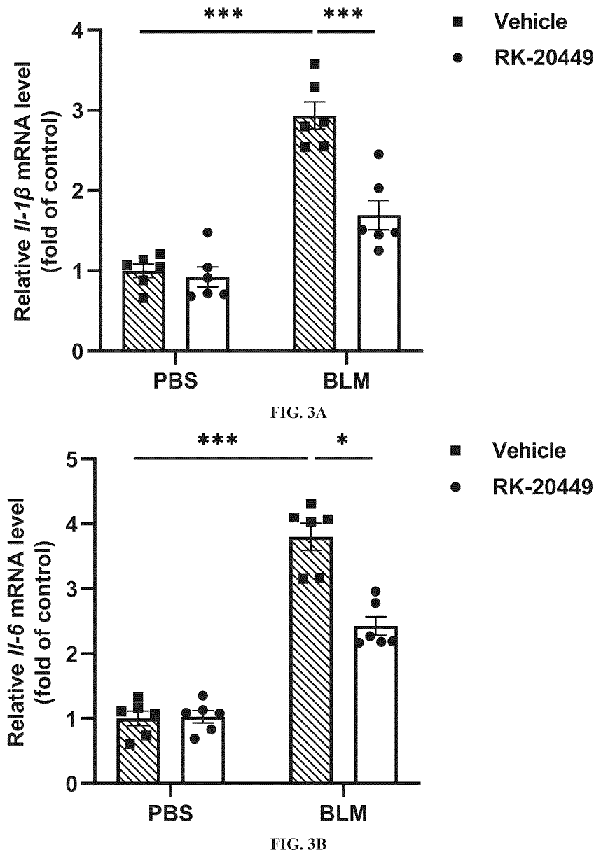

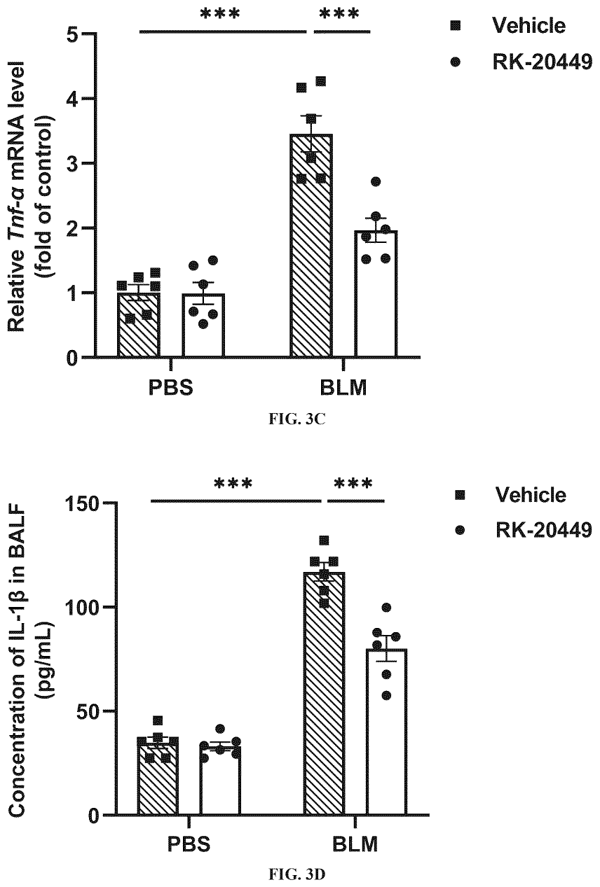

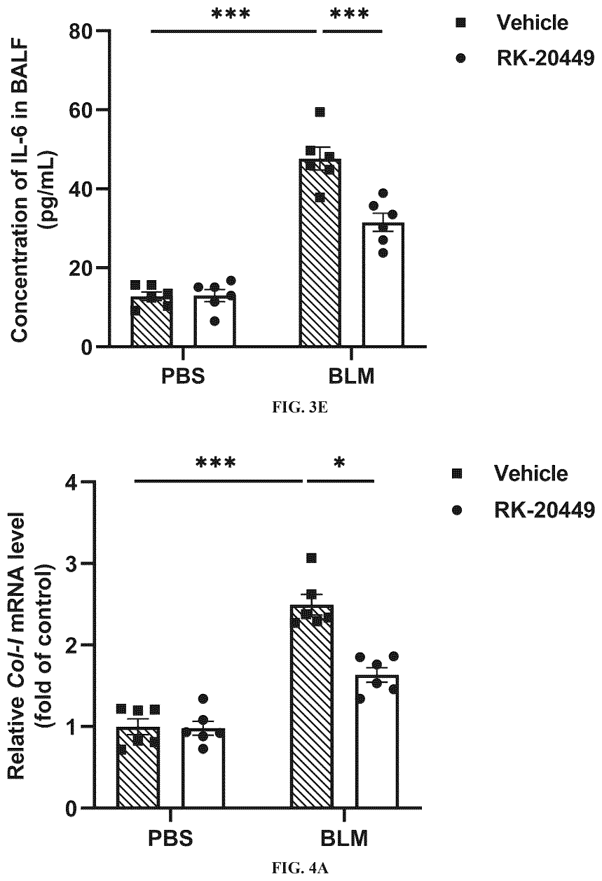

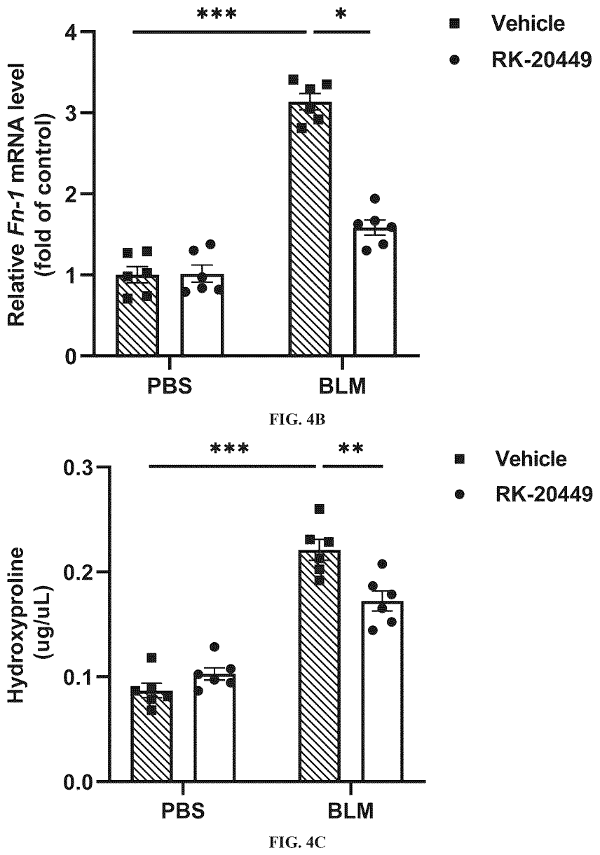

The results were shown in FIG. 1. Administration of the HCK inhibitor RK-20449 to IPF mice could effectively inhibit the transcription level of Hck in the lung tissue of mice. In addition, RK-20449 could also alleviate the progression of pulmonary fibrosis. After administration of the HCK inhibitor RK-20449, the weight loss of IPF mice was alleviated (FIG. 2A), and the survival rate was also increased to a certain extent (FIG. 2B). The lung function of IPF mice improved significantly, including lung volume indicators such as IC (FIG. 2F), and lung ventilation function tests such as RI (FIG. 2C), Cdyn (FIG. 2D) and Cchord (FIG. 2E); the transcription levels of inflammatory factors Il-1β (FIG. 3A), Il-6 (FIG. 3B), and Tnf-α (FIG. 3C) were decreased in IPF mice; the concentrations of inflammatory factors IL-1β (FIG. 3D) and IL-6 (FIG. 3E) in the alveolar lavage fluid decreased. The transcription levels of fibrosis factors Col-I (FIG. 4A) and Fn-1 (FIG. 4B) decreased, the collagen-specific amino acid hydroxyproline (FIG. 4C) decreased, and the degree of lesions was alleviated.

As shown in FIG. 1 to FIGS. 4A-4C, RK20449 could inhibit the transcription level of Hck in mouse lung tissue and effectively alleviate the progression of pulmonary fibrosis. Administration of the HCK inhibitor RK20449 to BLM mice could significantly improve the lung function, inflammation and fibrosis symptoms of IPF mice, indicating that HCK had important regulatory functions and targeting significance in the progression of pulmonary fibrosis. Therefore, the HCK inhibitor can be used as a new treatment option for pulmonary fibrosis, especially for the treatment of idiopathic pulmonary fibrosis.

The above descriptions are merely preferred implementations of the present disclosure. It should be noted that a person of ordinary skill in the art may further make several improvements and modifications without departing from the principle of the present disclosure, but such improvements and modifications should be deemed as falling within the protection scope of the present disclosure.

What is claimed is:

1. A method for preparation of a drug for treating pulmonary fibrosis using a hematopoietic cell kinase (HCK) inhibitor.

2. The method according to claim 1, wherein the HCK inhibitor is one or two selected from the group consisting of a modulator capable of reducing an HCK expression level and a modulator capable of reducing an HCK product.

3. The method according to claim 2, wherein the modulator capable of reducing the HCK expression level comprises RK-20449.

4. The method according to claim 3, wherein an RK-20449 solution has a concentration of 5 mg/mL to 15 mg/mL.

5. The method according to claim 4, wherein the RK-20449 solution is prepared with phosphate-buffered saline (PBS) as a solvent.

6. The method according to claim 2, wherein the HCK inhibitor comprises a protease and a nuclease that degrade the HCK product.

7. The method according to claim 1, wherein the drug ameliorates pulmonary dysfunction.

8. The method according to claim 2, wherein the drug ameliorates pulmonary dysfunction.

9. The method according to claim 3, wherein the drug ameliorates pulmonary dysfunction.

10. The method according to claim 4, wherein the drug ameliorates pulmonary dysfunction.

11. The method according to claim 5, wherein the drug ameliorates pulmonary dysfunction.

12. The method according to claim 6, wherein the drug ameliorates pulmonary dysfunction.

13. The method according to claim 1, wherein the drug ameliorates pulmonary inflammation and pulmonary fibrosis.

14. The method according to claim 2, wherein the drug ameliorates pulmonary inflammation and pulmonary fibrosis.

15. The method according to claim 3, wherein the drug ameliorates pulmonary inflammation and pulmonary fibrosis.

16. The method according to claim 4, wherein the drug ameliorates pulmonary inflammation and pulmonary fibrosis.

17. The method according to claim 5, wherein the drug ameliorates pulmonary inflammation and pulmonary fibrosis.

18. The method according to claim 6, wherein the drug ameliorates pulmonary inflammation and pulmonary fibrosis.

19. A drug for treating pulmonary fibrosis, comprising an active ingredient and a pharmaceutically acceptable carrier, wherein the active ingredient comprises the modulator RK-20449 that reduces the HCK expression level according to claim 3.

20. The drug according to claim 19, wherein a dosage form of the drug is selected from the group consisting of a capsule, a powder, a tablet, and a solution.
